(19) United States
(12) Patent Application Publication
(10) Pub. No.: US 2024/0252469 A1
(43) Pub. Date: Aug. 1, 2024

(54) Carnosine Analogs for Use in the Treatment of Metabolic Disorders

(71) Applicant: Nottingham Trent University, Nottinghamshire (GB)

(72) Inventors: Mark Duncan Turner, Nottinghamshire (GB); Alun Christopher Garner, Nottinghamshire (GB)

(21) Appl. No.: 18/578,664

(22) PCT Filed: Jul. 8, 2022
(86) PCT No.: PCT/GB2022/051770

§ 371 Date: Jan. 11, 2024

(30) Foreign Application Priority Data
Jul. 15, 2021 (GB) 2110229.8

(51) Int. Cl. A61K 31/4172; A61P 3/10

(52) U.S. Cl. CPC A61K 31/4172; A61P 3/10

(57) ABSTRACT

The invention provides a compound of Formula (I) or a tautomer, isomer, prodrug, metal complex, or pharmaceutically acceptable salt thereof for use in the treatment of a metabolic disorder, wherein R¹ is: a straight or branched C₁-C₂₀ alkoxy, preferably C₁-C₁₀ alkoxy group, optionally containing one or more rings, and/or optionally containing one or more double bonds; an alkoxycarbonyloxyalkoxy group bearing a straight, branched or cyclic alkyl; an aryloxy group; or an arylalkoxy group; R² and R³ which can be the same or different, are: hydrogen; a straight or branched C₁-C₂₀ alkylcarbonyl or cyclic C₃-C₇ alkylcarbonyl group optionally containing one or more double bonds; an arylcarbonyl or arylalkylcarbonyl group; a straight or branched C₁-C₁₀ alkoxycarbonyl or cyclic C₃-C₇ alkoxycarbonyl group optionally containing one or more double bonds; an arylalkoxy carbonyl group; an amino group; a hydroxy group; or a group of general Formula (II) wherein Y is nitrogen, oxygen or sulfur and A is hydrogen or an amino group.

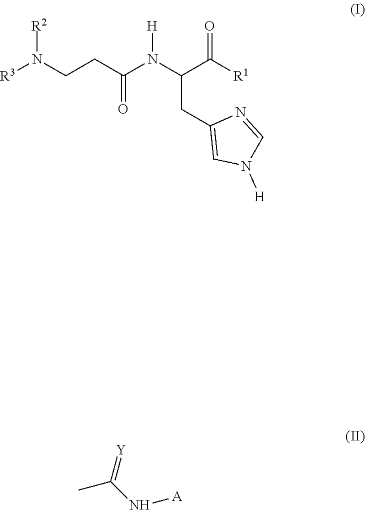

### TECHNICAL FIELD OF THE INVENTION

The present invention relates to carnosine esters for use in the treatment of metabolic disorders, uses of carnosine esters in the manufacture of a medicament for metabolic disorders, and to methods of medical treatment comprising the use of carnosine esters to treat metabolic disorders.

### BACKGROUND TO THE INVENTION

Diabetes is a group of chronic metabolic disorders characterised by high levels of glucose in the blood. The prevalence is rapidly increasing globally, and the World Health Organisation reported that 422 million people were living with diabetes in 2014, resulting to 1.6 million deaths due to diabetes in 2016. If more effective interventions are not developed, it is estimated that 629 million people will be diabetic in 2045, with enormous global economic burden and reduction in the quality adjusted life and years of diabetic patients. Of these, ˜95% have type-2 diabetes. Furthermore, whilst the development of therapies for type-2 diabetes treatment/management has improved since the first therapeutic interventions in the 1950's, their effectiveness typically diminishes over time. Crucially therefore, type-2 diabetes remains a 21st century global health challenge, with an urgent and currently unmet clinical need to develop more effective and novel therapies.

Approximately 80% of all people with type-2 diabetes are also obese. The fatty acids associated with obesity combine with glucose and its breakdown products to form damaging non-enzymatic glycation and lipidation end-products that bind to protein, lipid, and DNA, thereby modifying them and preventing normal cellular function.

Previous work indicates that carnosine, a naturally occurring physiological dipeptide, is an effective scavenger of glycation and lipidation end-products, and consequently is able to restore cellular function in key tissues associated with both insulin secretion (pancreatic β-cells) and insulin resistance (skeletal muscle) (Cripps, M. J., Hanna, K., Lavilla, C., Sayers, S. R., Caton, P. W., Sims, C., De Girolamo, L., Sale, C. and Turner, M. D., 2017. Carnosine scavenging of glucolipotoxic free radicals enhances insulin secretion and glucose uptake. Scientific reports, 7(1), pp. 1-7). However, taking carnosine as a supplement is likely to require sustained administration of high doses in order to achieve modest beneficial effects, as there are carnosinase enzymes in both blood and tissues that are able to degrade carnosine.

There exists a need for therapeutics that retain or even expand upon the beneficial biological actions of carnosine, but which also display limited toxicity and resistance to enzymatic degradation, particularly by carnsosinases.

It is an aim of embodiments of the present invention to address these requirements by providing compounds which provide one or more of the following advantages:
Non-degradability or slow degradability by carnosinase enzymes and/or improved bioavailability.
Ability to be used in the treatment of at least one metabolic disorder.
Ability to be used in the treatment of a weight or dietary-related metabolic disorder.
Ability to assist in regulating blood glucose levels and/or the ability to be used in the treatment of glucose-related metabolic disorders, such as diabetes; and preferably type-2 diabetes.
Limited or no toxicity.
Reactive species scavenging ability.
Membrane transport compatibility.

It is also an aim of embodiments of the invention to overcome or mitigate at least one problem of the prior art, whether expressly described herein or not.

### SUMMARY OF THE INVENTION

According to a first aspect of the invention, there is provided a compound of Formula (I) or a tautomer, isomer, prodrug, metal complex, or pharmaceutically acceptable salt thereof for use in the treatment of a metabolic disorder:

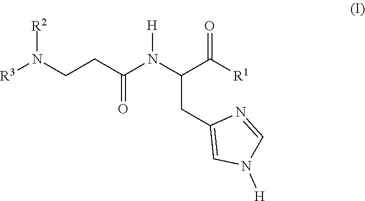

wherein R¹ is:
a straight or branched C₁-C₂₀ alkoxy, preferably C₁-C₁₀ alkoxy group, optionally containing one or more rings, and/or optionally containing one or more double bonds; an alkoxycarbonyloxyalkoxy group bearing a straight, branched or cyclic alkyl; an aryloxy group; or an arylalkoxy group;
R² and R³ which can be the same or different, are:
hydrogen; a straight or branched C₁-C₂₀ alkylcarbonyl or cyclic C₃-C₇ alkylcarbonyl group optionally containing one or more double bonds; an arylcarbonyl or arylalkylcarbonyl group; a straight or branched C₁-C₁₀ alkoxycarbonyl or cyclic C₃-C₇ alkoxycarbonyl group optionally containing one or more double bonds; an arylalkoxycarbonyl group; an amino group; a hydroxy group; or a group of general Formula (II)

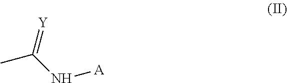

wherein Y is nitrogen, oxygen or sulfur and A is hydrogen or an amino group.

Such modified carnosine derivatives retain beneficial biological actions of carnosine, whilst being less quickly degraded by carnosinase enzymes. Such compounds are suitable for use as therapeutics in the treatment of metabolic disorders, and in particular weight or dietary-related metabolic disorders. Such compounds also assist in regulating blood glucose levels and may be especially useful in the treatment of glucose-related metabolic disorders, such as diabetes.

The aryl moieties of the aryloxy, arylalkoxy, arylcarbonyl, arylalkylcarbonyl, and arylalkoxycarbonyl groups defined above may be mono- or polycyclic and optionally substituted with one or more substituents selected from the group comprising: hydroxy; C₁-C₅-alkoxy; C₁-C₅-alkoxycarbonyl; amino; C₁-C₅-mono- or di-alkylamino; C₁-C₅-acylamino; halogen such as Cl, Br, F, and I; straight, branched or cyclic alkyl; optionally substituted aryl.

The aryl moieties may comprise phenyl or naphthyl, optionally substituted with one or more substituents selected from the group comprising: hydroxy, methyl, cyclopropyl, methoxy, amino, dimethylamino, methylamino, ethylamino, diethylamino, acetylamino, formylamino, propionylamino, butanoylamino, and halogen.

The alkyl residue of the alkoxy, alkoxycarbonyloxyalkoxy, arylalkoxy, alkylcarbonyl, arylalkylcarbonyl, alkoxycarbonyl, and arylalkoxycarbonyl groups defined above may be selected from the group comprising: methyl, ethyl, propyl, iso-propyl, n-butyl, sec-butyl, tert-butyl, n-pentyl, n-hexyl, n-octyl, n-decyl, and n-hexadecyl.

The cyclic alkyl residue may be selected from the group comprising: cyclopropyl, cyclopentyl, and cyclohexyl.

In some embodiments, R¹ is an arylalkoxy group; and R² and R³ which can be the same or different, are: hydrogen; a straight or branched C₁-C₂₀ alkylcarbonyl or cyclic C₃-C₇ alkylcarbonyl group optionally containing one or more double bonds; an arylcarbonyl or arylalkylcarbonyl group; a straight or branched C₁-C₁₀ alkoxycarbonyl or cyclic C₃-C₇ alkoxycarbonyl group optionally containing one or more double bonds; an arylalkoxycarbonyl group; an amino group; a hydroxy group; or a group of general Formula (II) wherein Y is nitrogen, oxygen or sulfur and A is hydrogen or an amino group.

In some embodiments, R¹ is an aryloxy group; and R² and R³ which can be the same or different, are: hydrogen; a straight or branched C₁-C₂₀ alkylcarbonyl or cyclic C₃-C₇ alkylcarbonyl group optionally containing one or more double bonds; an arylcarbonyl or arylalkylcarbonyl group; a straight or branched C₁-C₁₀ alkoxycarbonyl or cyclic C₃-C₇ alkoxycarbonyl group optionally containing one or more double bonds; an arylalkoxycarbonyl group; an amino group; a hydroxy group; or a group of general Formula (II) wherein Y is nitrogen, oxygen or sulfur and A is hydrogen or an amino group.

In some embodiments, R¹ is an alkoxycarbonyloxyalkoxy group bearing a straight, branched or cyclic alkyl; and R² and R³ which can be the same or different, are: hydrogen; a straight or branched C₁-C₂₀ alkylcarbonyl or cyclic C₃-C₇ alkylcarbonyl group optionally containing one or more double bonds; an arylcarbonyl or arylalkylcarbonyl group; a straight or branched C₁-C₁₀ alkoxycarbonyl or cyclic C₃-C₇ alkoxycarbonyl group optionally containing one or more double bonds; an arylalkoxycarbonyl group; an amino group; a hydroxy group; or a group of general Formula (II) wherein Y is nitrogen, oxygen or sulfur and A is hydrogen or an amino group.

R¹ is preferably alkoxy. Alkoxy R¹ groups have been found to provide the most clinically useful molecules of Formula (I), especially in the treatment or management of diabetes.

In some embodiments, R¹ is a straight or branched C₁-C₂₀ alkoxy, optionally containing one or more rings, and/or optionally containing one or more double bonds.

In some embodiments, R¹ is a straight or branched C₁-C₂₀ alkoxy.

In some embodiments, R¹ is a straight or branched C₁-C₁₀ alkoxy, optionally containing one or more rings, and/or optionally containing one or more double bonds.

In some embodiments, R¹ is a straight or branched C₁-C₁₀ alkoxy.

In some embodiments, R¹ is a straight or branched C₁-C₈ alkoxy, optionally containing one or more rings, and/or optionally containing one or more double bonds.

In some embodiments, R¹ is a straight or branched C₁-C₈ alkoxy.

In some embodiments, R¹ is a straight or branched C₁-C₆ alkoxy, optionally containing one or more rings, and/or optionally containing one or more double bonds.

In some embodiments, R¹ is a straight or branched C₁-C₆ alkoxy.

In some embodiments, R¹ is a straight or branched C₁-C₅ alkoxy, optionally containing a ring, and/or optionally containing one or more double bonds.

In some embodiments, R¹ is a straight or branched C₁-C₅ alkoxy.

In some embodiments, R¹ is a straight or branched C₁-C₄ alkoxy, optionally containing a ring, and/or optionally containing one or two double bonds.

In some embodiments, R¹ is a straight or branched C₁-C₄ alkoxy.

In preferred embodiments, R¹ is a straight or branched C₁-C₃ alkoxy, optionally containing a ring, and/or optionally containing a double bond.

In some preferred embodiments, R¹ is a straight or branched C₁-C₃ alkoxy.

In some preferred embodiments, R¹ is a straight or branched C₃ alkoxy, optionally containing a ring, and/or optionally containing a double bond.

In some embodiments, R¹ is a straight or branched C₃ alkoxy.

In some preferred embodiments, R¹ is C₂ alkoxy, optionally containing a double bond.

In some preferred embodiments, R¹ is C₂ alkoxy.

In some preferred embodiments, R¹ is C₁ alkoxy.

In some embodiments, R² and R³ are the same.

In preferred embodiments, R² and R³ are both hydrogen.

In some embodiments, R¹ is a straight or branched C₁-C₂₀ alkoxy, optionally containing one or more rings, and/or optionally containing one or more double bonds; and R² and R³ are the same, and preferably both hydrogen.

In some embodiments, R¹ is a straight or branched C₁-C₂₀ alkoxy; and R² and R³ are the same, and preferably both hydrogen.

In some embodiments, R¹ is a straight or branched C₁-C₁₀ alkoxy, optionally containing one or more rings, and/or optionally containing one or more double bonds; and R² and R³ are the same, and preferably both hydrogen.

In some embodiments, R¹ is a straight or branched C₁-C₁₀ alkoxy; and R² and R³ are the same, and preferably both hydrogen.

In some embodiments, R¹ is a straight or branched C₁-C₈ alkoxy, optionally containing one or more rings, and/or optionally containing one or more double bonds; and R² and R³ are the same, and preferably both hydrogen.

In some embodiments, R¹ is a straight or branched C₁-C₈ alkoxy; and R² and R³ are the same, and preferably both hydrogen.

In some embodiments, R¹ is a straight or branched C₁-C₆ alkoxy, optionally containing one or more rings, and/or optionally containing one or more double bonds; and R² and R³ are the same, and preferably both hydrogen.

In some embodiments, R¹ is a straight or branched C₁-C₆ alkoxy; and R² and R³ are the same, and preferably both hydrogen.

In some embodiments, R¹ is a straight or branched C₁-C₅ alkoxy, optionally containing a ring, and/or optionally containing one or more double bonds; and R² and R³ are the same, and preferably both hydrogen.

In some embodiments, R¹ is a straight or branched C₁-C₅ alkoxy; and R² and R³ are the same, and preferably both hydrogen.

In some embodiments, R¹ is a straight or branched C₁-C₄ alkoxy, optionally containing a ring, and/or optionally containing one or two double bonds; and R² and R³ are the same, and preferably both hydrogen.

In some embodiments, R¹ is a straight or branched C₁-C₄ alkoxy; and R² and R³ are the same, and preferably both hydrogen.

In preferred embodiments, R¹ is a straight or branched C₁-C₃ alkoxy, optionally containing a ring, and/or optionally containing a double bond; and R² and R³ are the same, and preferably both hydrogen.

In preferred embodiments, R¹ is a straight or branched C₁-C₃ alkoxy; and R² and R³ are the same, and preferably both hydrogen.

In some preferred embodiments, R¹ is a straight or branched C₃ alkoxy, optionally containing a ring, and/or optionally containing a double bond; and R² and R³ are the same, and preferably both hydrogen.

In some preferred embodiments, R¹ is a straight or branched C₃ alkoxy; and R² and R³ are the same, and preferably both hydrogen.

In some preferred embodiments, R¹ is C₂ alkoxy, optionally containing a double bond; and R² and R³ are the same, and preferably both hydrogen.

In some preferred embodiments, R¹ is C₂ alkoxy; and R² and R³ are the same, and preferably both hydrogen.

In some preferred embodiments, R¹ is C₁ alkoxy; and R² and R³ are the same, and preferably both hydrogen.

In some embodiments, R¹ is an alkoxy group comprising an alkyl residue selected from the group comprising: methyl, ethyl, propyl, iso-propyl, n-butyl, see-butyl, tert-butyl, n-pentyl, n-hexyl, n-octyl, n-decyl, and n-hexadecyl.

In some embodiments, R¹ is an alkoxy group comprising an alkyl residue selected from the group comprising: methyl, ethyl, propyl, iso-propyl, n-butyl, see-butyl, tert-butyl, n-pentyl, n-hexyl, n-octyl, n-decyl, and n-hexadecyl; and R² and R³ are the same, and preferably both hydrogen.

In preferred embodiments, R¹ is an alkoxy group comprising an alkyl residue selected from the group comprising: methyl, ethyl, and iso-propyl.

In preferred embodiments, R¹ is an alkoxy group comprising an alkyl residue selected from the group comprising: methyl, ethyl, and iso-propyl; and R² and R³ are the same, and preferably both hydrogen.

In preferred embodiments, the compound is selected from the group comprising: carnosine methyl ester, carnosine ethyl ester, and carnosine iso-propyl ester, or a tautomer, isomer, prodrug, metal complex, or pharmaceutically acceptable salt thereof.

In an especially preferred embodiment, the compound is carnosine iso-propyl ester or a tautomer, isomer, prodrug, metal complex, or pharmaceutically acceptable salt thereof.

In preferred embodiments, the compound is the L isomer. The L isomer is believed to provide improved biological activity.

In preferred embodiments, the compound is present as a hydrochloride salt, preferably a dihydrochloride salt.

The compounds of the invention can be prepared using well known techniques, starting from L-histidine and 3-aminopropionic intermediates containing the residues R¹, R², and R³ as defined above, or starting from 3-aminopropionyl-L-histidine for the subsequent introduction of the necessary substituents.

For example, the compounds of the invention can be prepared by coupling 3-aminopropionic derivatives, suitably substituted at the nitrogen with L-histidine derivatives bearing the appropriate R¹ substituent, optionally suitably protected, using a coupling method as those described for example in Houben Weil “Synthesis of peptides and peptidomimetics” E 22a chapter 3.

Alternatively, the compounds can be prepared starting from 3-aminopropionyl-L-histidine by subsequent introduction of the necessary substituents using established procedures, such as those reported in T. Greene, P. Wuts “Protective Groups in Organic Chemistry” for functionalisation of the carboxylic and amino groups.

For the intended uses, the compound may be formulated in a conventional pharmaceutical, cosmetic, or nutritional composition. The composition may be suitable for administration orally, parenterally, topically, or transdermally. The composition may comprise a solid, a capsule, tablet, syrup, injectable solution or suspension, ointment, suppository, controlled-release form, water-soluble granulate. The composition may comprise other active ingredients having complementary or anyway useful activity in addition to the carriers and excipients used in the pharmaceutical technique. The composition may contain cinnamon and/or chromium.

The compound may comprise a dosage of at least 1 mg/kg body weight/day, or of at least 2, 3, 4, 5, 6, 7, 8, 9, 10, 15, 20, 25, 30, 35, or of at least 40 mg/kg body weight/day. The compound may comprise a dosage of no greater than 100 mg/kg body weight/day, or of no greater than 95, 90, 85, 80, 75, 70, 65, 60, 55, or of no greater than 50 mg/kg body weight/day. The compound may preferably comprise a dosage of between 40-50 mg/kg body weight/day.

In some embodiments, the metabolic disorder comprises a weight or dietary-related metabolic disorder. The weight or dietary-related metabolic disorder may be selected from the group comprising: a glucose-related metabolic disorder, obesity, dyslipidemia, hypertension, and metabolic syndrome.

In some embodiments, the metabolic disorder comprises a glucose-related metabolic disorder. The glucose-related metabolic disorder may be selected from the group comprising: type-1 diabetes, type-2 diabetes, prediabetes, insulin resistance, impaired glucose tolerance, elevated blood glucose, hyperinsulinemia, and diabetes related diseases.

In preferred embodiments, the metabolic disorder comprises type-2 diabetes.

It has been found that carnosine methyl ester, carnosine ethyl ester, and carnosine iso-propyl ester, or a tautomer, isomer, prodrug, metal complex, or pharmaceutically acceptable salt thereof are particularly effective against diabetes, particularly type-2 diabetes.

According to a second aspect of the invention, there is provided the use of a compound of Formula (I) or a tautomer, isomer, prodrug, metal complex, or pharmaceutically acceptable salt thereof in the manufacture of a medicament for a metabolic disorder.

The compound may comprise any compound of the first aspect of the invention.

The metabolic disorder may comprise any metabolic disorder of the first aspect of the invention.

According to a third aspect of the invention, there is provided a method of treating a metabolic disorder in a subject in need of treatment with a compound of Formula (I) or a tautomer, isomer, prodrug, metal complex, or pharmaceutically acceptable salt thereof.

The compound may comprise any compound of the first aspect of the invention.

The metabolic disorder may comprise any metabolic disorder of the first aspect of the invention.

The method may comprise administering the compound at a dosage of at least 1 mg/kg body weight/day, or of at least 2, 3, 4, 5, 6, 7, 8, 9, 10, 15, 20, 25, 30, 35, or of at least 40 mg/kg body weight/day. The method may comprise administering the compound at a dosage of no greater than 100 mg/kg body weight/day, or of no greater than 95, 90, 85, 80, 75, 70, 65, 60, 55, or of no greater than 50 mg/kg body weight/day. The method may preferably comprise administering the compound at a dosage of between 40-50 mg/kg body weight/day.

### DETAILED DESCRIPTION OF THE INVENTION

In order that the invention may be more clearly understood embodiments thereof will now be described, by way of example only, with reference to the accompanying drawings, of which:

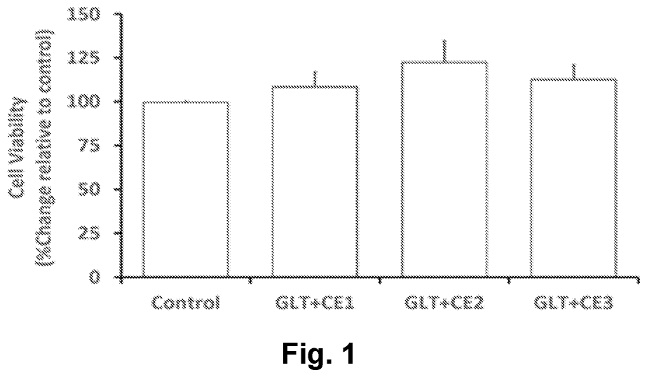

FIG. 1 shows a bar graph displaying cell viability (expressed as a % change relative to control from 3 independent experiments±SEM) of C2C12 skeletal muscle cells following their culture in four different media for 5 days. Bars represent the following media used: Control—a Roswell Park Memorial Institute-1640 (RPMI-1640) control medium; GLT+CE1—a glucolipotoxic (GLT) RPMI-1640 medium (28 mM glucose, 200 μM palmitic acid and 200 μM oleic acid) with 100 μM of added L-carnosine methyl ester; GLT+CE2—GLT RPMI-1640 medium with 100 μM of added L-carnosine ethyl ester; and GLT+CE3—GLT RPMI-1640 medium with 100 μM of added L-carnosine iso-propyl ester. The graph shows that none of the three carnosine esters reduce C2C12 skeletal muscle cell viability.

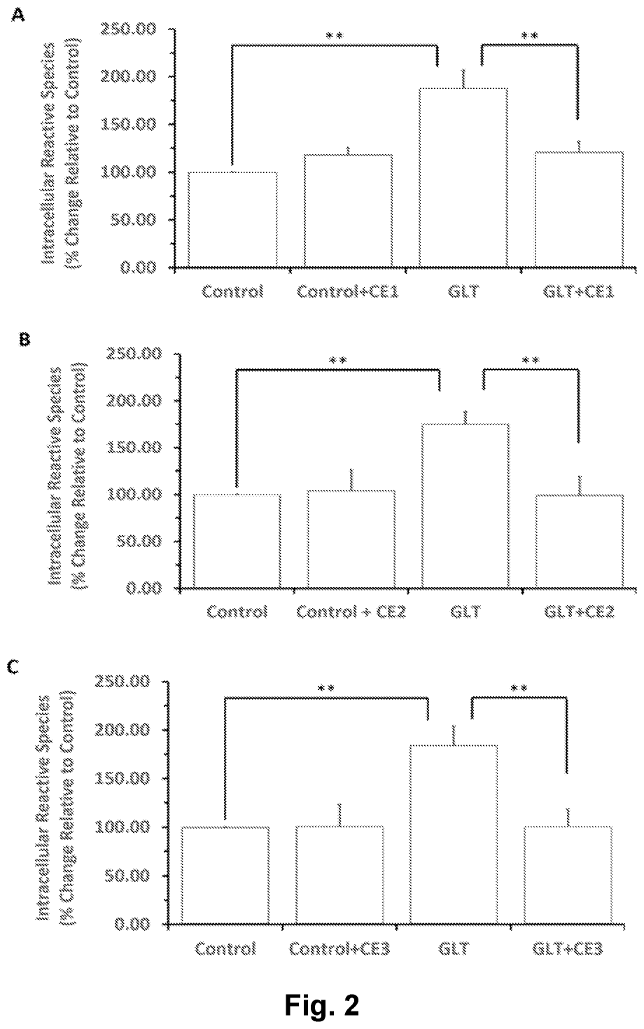

FIG. 2 shows bar graphs (A, B, and C) displaying reactive species scavenging abilities of three carnosine esters (A—L-carnosine methyl ester (CE1), B L-carnosine ethyl ester (CE2), and C—L-carnosine iso-propyl ester (CE3)) that were independently used to treat for 1 h periods C2C12 skeletal muscle cells that had been cultured in control RPMI-1640 or GLT RPMI-1640 media for 5 days. Graphs show intracellular reactive species (expressed as a % change relative to control from 3 or 4 independent experiments±SEM; ** represents a change with p<0.005). Bars represent from left to right: Control—cells cultured in control medium with no additional compounds added; Control+CE compound—cells cultured in control medium and treated with one of the three carnosine esters; GLT—cells cultured in GLT medium with no additional compounds added; and GLT+CE compound—cells cultured in GLT medium and treated with one of the three carnosine esters. The graphs show that exposure of cells to GLT media results in significantly enhanced presence of reactive species within the cells. Independent incubation in the presence of the carnosine esters causes GLT-associated reactive species levels to return to control values.

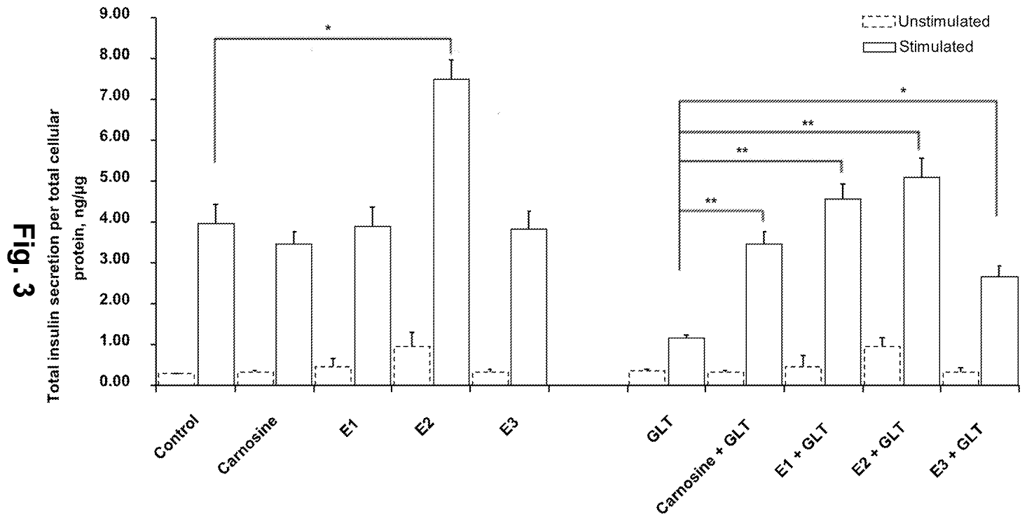

FIG. 3 shows a bar graph displaying insulin secretion from INS-1 pancreatic β-cells treated with selected carnosine esters. The graph shows total insulin secretion per total cellular protein (ng/μg) of INS-1 cells that were cultured for 5 days in various RPMI-1640 media. Data is shown for cells in which insulin secretion was stimulated through 2 h treatment with secretagogue cocktail as well as unstimulated cells. Bars represent the following media used: Control—a control RPMI-1640 medium; Carnosine—RPMI-1640 medium supplemented with 10 mM of L-carnosine; E1—RPMI-1640 medium supplemented with 100 μM of L-carnosine methyl ester; E2—RPMI-1640 medium supplemented with 100 μM of L-carnosine ethyl ester; E3—RPMI-1640 medium supplemented with 100 μM of L-carnosine iso-propyl ester; GLT—GLT RPMI-1640 medium; Carnosine+GLT—GLT RPMI-1640 medium supplemented with 10 mM of L-carnosine; E1+GLT—GLT RPMI-1640 medium supplemented with 100 μM of L-carnosine methyl ester; E2+GLT—GLT RPMI-1640 medium supplemented with 100 μM of L-carnosine ethyl ester; E3+GLT—GLT RPMI-1640 medium supplemented with 100 μM of L-carnosine iso-propyl ester. Data are expressed as mean±SEM from 3 independent experiments. ** represents a change with p<0.005 and * represents a change with p<0.05. The graph demonstrates the ability of the carnosine esters to reverse glucolipotoxic inhibition of insulin secretion; an action beneficial to the control of glucose homeostasis and the control of blood sugar levels in type-2 diabetes.

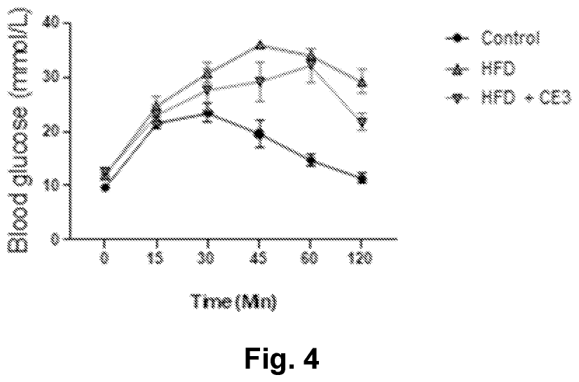

FIG. 4 shows a graph of blood glucose (mmol/L) versus time after a glucose solution was administered to spontaneously diabetic high fat-fed mice. The graph contains three curves: a Control curve wherein the glucose solution was administered to non-diabetic, non-high fat-fed mice; an HFD curve wherein the glucose solution was administered to spontaneously diabetic high fat-fed mice without administration of any other compound; and an HFD+CE3 curve wherein both the glucose solution and L-carnosine iso-propyl ester were administered to spontaneously diabetic high fat-fed mice. The graph shows that administration of L-carnosine iso-propyl ester allows for significantly improved glucose tolerance in spontaneously diabetic high fat-fed mice.

### EXAMPLES

All experiments were approved by a local ethical review committee and carried out under UK Home Office approval and according to the Animals Scientific Procedures Act (1986).

All carnosine esters were used as their hydrochloride salts.

### Toxicity Testing of Carnosine Esters

C2C12 muscle cell myotubes were cultured for 5 days in the following four culture media: a control RPMI-1640 medium; glucolipotoxic (GLT) RPMI-1640 medium (28 mM glucose, 200 μM palmitic acid and 200 μM oleic acid) supplemented with 100 μM of L-carnosine methyl ester; GLT RPMI-1640 medium supplemented with 100 μM of L-carnosine ethyl ester; and GLT RPMI-1640 medium supplemented with 100 μM of L-carnosine iso-propyl ester. Following culture, media were aspirated and cells washed 3 times in Krebs-Ringer buffer (KRB). A final concentration of 5 μM Calcein AM Cell Viability Dye (ThermoFischer) in KRB was loaded for 1 h before washing again with KRB. Cell viability was measured via fluorescence, with excitation and emission at 490 nm and 520 nm, respectively.

### Results of Toxicity Testing of Carnosine Esters

The results of the carnosine ester toxicity testing, as displayed in FIG. 1, show that none of the three carnosine esters (L-carnosine methyl ester, L-carnosine ethyl ester, and L-carnosine iso-propyl ester) reduce C2C12 skeletal muscle cell viability and are thus non-toxic to the cells.

### Reactive Species Scavenging Ability of Carnosine Esters

C2C12 muscle cell myotubes were cultured for 5 days in standard RPMI-1640 tissue culture media or GLT RPMI-1640 media. Corresponding media were then replaced and supplemented independently with 100 μM of a carnosine ester (L-carnosine methyl ester, L-carnosine ethyl ester, and L-carnosine iso-propyl ester investigated) for 1 h. Non-supplemented standard and GLT RPMI-1640 media were also retained as controls. Cells were thereafter washed 3 times in KRB and 20 μM 2′,7′-dichlorofluorescein diacetate (DCFDA), a cell permeant fluorogenic dye that measures hydroxyl, peroxyl and other ROS, was loaded for 1 h. Reactive species detection was measured via fluorescence, with excitation at 495 nm and emission at 530 nm.

### Results of Reactive Species Scavenging Ability of Carnosine Esters

The results of the carnosine ester reactive species scavenging testing, as displayed in FIG. 2, show that exposure to glucolipotoxic (GLT) media results in significantly enhanced presence of damaging reactive species within cells.

Incubation in the presence of the indicated carnosine ester reduced GLT-associated reactive species levels back down to non-GLT control levels.

The reactive species scavenging ability of the carnosine esters potentially confers significant clinical benefit to use of the compounds as therapeutics to treat diseases associated with metabolic stress. For instance, the ability to scavenge glycation and lipidation end-products allows for restoration of normal cellular function in key tissues associated with insulin secretion and insulin resistance.

Insulin Secretion from INS-1 Pancreatic β-Cells Treated with Carnosine Esters

INS-1 pancreatic B-cells were cultured for 5 days in standard RPMI-1640 tissue culture media or GLT RPMI-1640 media. The media were either used without further supplementation (used as controls) or were independently supplemented with either 10 mM of L-carnosine or with 100 μM of a carnosine ester (L-carnosine methyl ester, L-carnosine ethyl ester, and L-carnosine iso-propyl ester investigated). INS-1 cells were then treated with KRB or KRB supplemented with secretagogue cocktail (13.5 mM glucose, 1 μM phorbol 12-myristate 13-acetate, 1 mM isobutyl-methylxanthine, 1 mM tolbutamide, 10 mM leucine, 10 mM glutamine) as a stimulant of insulin secretion for 2 h. Insulin secretion was determined by ELISA assay, with data normalised to cellular protein content.

Results of Insulin Secretion from INS-1 Pancreatic β-Cells Treated with Carnosine Esters

The results of insulin secretion from INS-1 cells treated with carnosine esters, as displayed in FIG. 3, show that 5-day exposure of the cells to GLT media results in significant reduction in secretagogue-stimulated insulin secretion.

Incubation of the cells in the presence of the indicated carnosine ester significantly increases insulin secretion in cells exposed to glucolipotoxicity (GLT), returning secretion close to, or above, control values. In a few cases, the carnosine esters even display improved activity in increasing insulin secretion compared to L-carnosine.

The 5-day exposure of INS-1 pancreatic β-cells to GLT media provides for a cellular model of type-2 diabetes, which is characterised by a significant reduction in insulin secretion caused by pancreatic dysfunction, which arises as the pancreas struggles to address a sustained demand for increased insulin secretion placed upon it. The ability of the carnosine esters to reverse GLT inhibition of insulin secretion is beneficial to the control of glucose homeostasis and the control of blood sugar levels in diseases such as type-2 diabetes, obesity, Metabolic Syndrome, and other associated diseases where cells and tissues are under sustained metabolic stress.

### Effect of L-Carnosine Iso-Propyl Ester on Glucose Tolerance in an In Vivo Model of Type-2 Diabetes

High fat-fed spontaneously diabetic mice were used as an animal model of type-2 diabetes. A glucose tolerance test was performed on three sets of mice, wherein the mice were initially fasted for 6 h. A 2 mg/kg glucose solution was then administered to the three sets of mice in their drinking water and their blood glucose concentrations (mmol/L) monitored over a period of 2 h. The following three sets of mice were used in the study:
Non-diabetic, non-high fat-fed mice.
Spontaneously diabetic high fat-fed mice.
Spontaneously diabetic high fat-fed mice whose diet also included 45 mg/kg body weight/day of L-carnosine iso-propyl ester.

Spontaneously diabetic high fat-fed mice were defined as mice which had been fed the following high fat diet from Research Diets: (60% fat; D12492). Non-diabetic, non-high fat-fed mice were used as a control and were fed the following low fat diet from Research Diets: (10% fat, D12450B). The body weights of the mice on the two different diets were measured throughout.

### Results of the Effect of L-Carnosine Iso-Propyl Ester on Glucose Tolerance in an In Vivo Model of Type-2 Diabetes

The results of the in vivo study, as displayed in FIG. 4, demonstrate that L-carnosine iso-propyl ester significantly improves glucose tolerance in the mice. The blood glucose concentrations at the end of the study (2 h) of high fat-fed mice that were administered L-carnosine iso-propyl ester are significantly lower than the corresponding high fat-fed mice that were only administered the high fat diet; with the concentration returning close to control values.

Furthermore, L-carnosine iso-propyl ester displayed no toxic effects on the animals.

It was found that mice which were only administered the high fat diet, without any carnosine ester, became insulin resistant and developed glucose intolerance by 8-10 weeks.

The results demonstrate the physiological benefit and efficacy of the hydrolytically stable carnosine esters in the regulation of blood glucose levels in living animals. The compounds are therefore useful in the treatment of glucose-related metabolic disorders, such as type-2 diabetes, obesity, Metabolic Syndrome, and other associated diseases where cells and tissues are under sustained metabolic stress.

The above embodiments are described by way of example only. Many variations are possible without departing from the scope of the invention as defined in the appended claims.

1. A compound of Formula (I) or a tautomer, isomer, prodrug, metal complex, or pharmaceutically acceptable salt thereof for use in the treatment of a metabolic disorder: 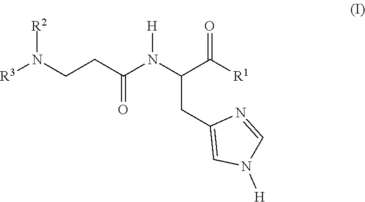
wherein R¹ is:
a straight or branched C₁-C₂₀ alkoxy, preferably C₁-C₁₀ alkoxy group, optionally containing one or more rings, and/or optionally containing one or more double bonds; an alkoxycarbonyloxyalkoxy group bearing a straight, branched or cyclic alkyl; an aryloxy group; or an arylalkoxy group;
R² and R³ which can be the same or different, are:
hydrogen; a straight or branched C₁-C₂₀ alkylcarbonyl or cyclic C₃-C₇ alkylcarbonyl group optionally containing one or more double bonds; an arylcarbonyl or arylalkylcarbonyl group; a straight or branched C₁-C₁₀ alkoxycarbonyl or cyclic C₃-C₇ alkoxycarbonyl group optionally containing one or more double bonds; an arylalkoxycarbonyl group; an amino group; a hydroxy group; or a group of general Formula (II) 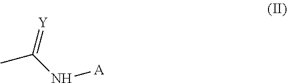
wherein Y is nitrogen, oxygen or sulfur and A is hydrogen or an amino group.

2. A compound as claimed in claim 1, wherein R² and R³ are the same.

3. A compound as claimed in claim 2, wherein R² and R³ are both hydrogen.

4. A compound as claimed in claim 1, wherein R¹ is a straight or branched C₁-C₁₀ alkoxy, preferably C₁-C₈ alkoxy group, optionally containing one or more rings, and/or optionally containing one or more double bonds.

5. A compound as claimed in claim 4, wherein R¹ is a straight or branched C₁-C₆ alkoxy group.

6. A compound as claimed in claim 4, wherein R¹ is a straight or branched C₁-C₄ alkoxy, preferably C₁-C₃ alkoxy group.

7. A compound as claimed in claim 1, wherein R¹ is an alkoxy group comprising an alkyl residue selected from the group comprising: methyl, ethyl, propyl, iso-propyl, n-butyl, sec-butyl, tert-butyl, n-pentyl, n-hexyl, n-octyl, n-decyl, and n-hexadecyl.

8. A compound as claimed in claim 1, wherein the compound is selected from the group comprising: carnosine methyl ester, carnosine ethyl ester, and carnosine iso-propyl ester, or a tautomer, isomer, prodrug, metal complex, or pharmaceutically acceptable salt thereof.

9. A compound as claimed in claim 8, wherein the compound is carnosine iso-propyl ester or a tautomer, isomer, prodrug, metal complex, or pharmaceutically acceptable salt thereof.

10. A compound as claimed in claim 1, wherein the compound is the L isomer.

11. A compound for use in the treatment of a metabolic disorder as claimed in claim 1, wherein the metabolic disorder comprises a weight or dietary-related metabolic disorder.

12. A compound for use in the treatment of a metabolic disorder as claimed in claim 11, wherein the weight or dietary-related metabolic disorder is selected from the group comprising: a glucose-related metabolic disorder, obesity, dyslipidemia, hypertension, and metabolic syndrome.

13. A compound for use in the treatment of a metabolic disorder as claimed in claim 12, wherein the glucose-related metabolic disorder is selected from the group comprising: type-1 diabetes, type-2 diabetes, prediabetes, insulin resistance, impaired glucose tolerance, elevated blood glucose, hyperinsulinemia, and diabetes related diseases.

14. A compound for use in the treatment of a metabolic disorder as claimed in claim 13, wherein the glucose-related metabolic disorder comprises type-2 diabetes.

15. Use of a compound of Formula (I) as defined in claim 1, or a tautomer, isomer, prodrug, metal complex, or pharmaceutically acceptable salt thereof in the manufacture of a medicament for a metabolic disorder.

16. A method of treating a metabolic disorder in a subject in need of treatment with a compound of Formula (I) as defined in claim 1, or a tautomer, isomer, prodrug, metal complex, or pharmaceutically acceptable salt thereof.
